(19) United States
(12) Patent Application Publication
(10) Pub. No.: US 2004/0229321 A1
(43) Pub. Date: Nov. 18, 2004

(54) Process for producing L-amino acids by fermentation of a mixture of glucose and pentoses

(76) Inventors: Ekaterina Alekseevna Savrasova, Moscow (RU); Elena Viktorovna Sycheva, Moscow (RU); Tatyana Anatolievna Michurina, Moscow (RU); Yuri Ivanovich Kozlov, Moscow (RU)

Correspondence Address: AJINOMOTO CORPORATE SERVICES, LLC, INTELLECTUAL PROPERTY DEPARTMENT, 1120 CONNECTICUT AVE., N.W., WASHINGTON, DC, 20036, US

(21) Appl. No.: 10/784,980

(22) Filed: Feb. 25, 2004

(30) Foreign Application Priority Data
Feb. 26, 2003 (RU) 2003105269

(51) Int. Cl. C12P 13/04; C12P 13/22; C12P 13/06; C12N 9/26; C12N 1/21

(52) U.S. Cl. 435/106; 435/116; 435/252.33; 435/108

(57) ABSTRACT

A process for producing an L-amino acid, such as L-isoleucine, L-histidine, L-threonine and L-tryptophan, using bacterium belonging to the genus Escherichia is provided which comprises cultivating the L-amino acid-producing bacterium in a culture medium and collecting the L-amino acid from the culture medium, wherein the culture medium contains a mixture of glucose and pentose sugars, such as arabinose and xylose.

### BACKGROUND OF THE INVENTION

1. Field of the Invention

The present invention relates to biotechnology, specifically to a process for producing L-amino acids by pentose fermentation and more specifically to a process for producing L-amino acids by fermentation using mixture of arabinose and/or xylose along with glucose as a carbon source. The non-expensive carbon source comprising the mixture of hexoses and pentoses of hemicellulose fractions from cellulosic biomass could be utilized for commercial production of L-amino acids, for example, L-isoleucine, L-histidine, L-threonine and L-tryptophan.

2. Description of the Related Art

Conventionally, L-amino acids have been industrially produced by a fermentation process using strains of different microorganisms. The fermentation media for the process should contain sufficient amounts of different sources of carbon and nitrogen.

Traditionally, various carbohydrates such as hexoses, pentoses, trioses; various organic acids and alcohols are used as a carbon source. Hexoses include glucose, fructose, mannose, sorbose, galactose and the like. Pentoses include arabinose, xylose, ribose and the like. But the above-mentioned carbohydrates and other traditional carbon sources, such as molasses, corn, sugarcane, starch, its hydrolysate etc. used in the industry are still rather expensive, and reducing the price of the resulting L-amino acids is desired.

Cellulosic biomass includes waste products from various bio-commercial processes, such as wood and grass, and is a favorable feedstock for L-amino acid production because it is both readily available and less expensive than carbohydrates, corn, sugarcane or other sources of carbon (http://www.ott.doe.gov/biofuels/glossary.html). The typical level of cellulose, hemicellulose and lignin in biomass is approximately 40-60% of cellulose, 20-40% of hemicellulose 10-25% of lignin and 10% of other components. The cellulose fraction consists of polymers of the hexose sugar, glucose. The hemicellulose fraction is comprised mostly of pentose sugars, including xylose and arabinose. Composition of various biomass feedstocks is shown in Table 1 (http://www.ott.doe.gov/biofuels/understanding_biomass.html.

TABLE 1

| | Six- | | | |
|---|---|---|---|---|
| Material | carbon sugars | Five-carbon sugars | Lignin | Ash |
| Hardwoods | 39-50% | 18-28% | 15-28% | 0.3-1.0% |
| Softwoods | 41-57% | 8-12% | 24-27% | 0.1-0.4% |

More detailed information about the composition of over 150 biomass samples is summarized in the “Biomass Feedstock Composition and Property Database” (http://www.ott.doe.gov/biofuels/progs/search1.cgi).

An industrial process for effective conversion of cellulosic biomass into usable fermentation feedstock (typically a mixture of carbohydrates) is expected to be developed in the very near future. Therefore, the utilization of renewable energy sources such as cellulose and hemicellulose for production of useful compounds is expected to increase in the nearest future (Aristidou A., Pentila.M., Curr. Opin. Biotechnol, 2000, Apr., 11:2, 187-198). But a great majority of published articles and patents (or published patent applications) describe the utilization of cellulosic biomass by biocatalysts (bacteria and yeasts) for production of ethanol, which is expected to be an alternative fuel. Such processes comprise fermentation of cellulosic biomass using different modified strains of Zymomonas mobilis (Deanda K. et al, Appl. Environ. Microbiol., 1996 Dec., 62:12, 4465-70; Mohagheghi A. et al, Appl. Biochem. Biotechnol., 2002, 98-100:885-98; Lawford H. G., Rousseau J. D., Appl. Biochem. Biotechnol, 2002, 98-100:429-48; PCT applications WO95/28476, WO98/50524), modified strains of Escherichia coli (Dien B. S. et al, Appl. Biochem. Biotechnol, 2000, 84-86:181-96; Nichols N. N. et al, Appl. Microbiol. Biotechnol., 2001 July, 56:1-2, 120-5; U.S. Pat. No. 5,000,000). Xylitol could be produced by fermentation of xylose from hemicellulosic sugars using Candida tropicalis (Walthers T. et al, Appl. Biochem. Biotechnol., 2001, 91-93:423-35). 1,2-propanediol could be produced by fermentation of arabinose, fructose, galactose, glucose, lactose, maltose, sucrose, xylose, and combination thereof using recombinant Escherichia coli strain (U.S. Pat. No. 6,303,352). Also it was shown that 3-dehydroshikimic acid could be obtained by fermentation of glucose/xylose/arabinose mixture using Escherichia coli strain and the highest concentrations and yields of 3-dehydroshikimic acid were obtained when the glucose/xylose/arabinose mixture was used as the carbon source relative to either xylose or glucose alone being used as a carbon source (Kai Li and J. W. Frost, Biotechnol. Prog., 1999, 15, 876-883). It is known that Escherichia coli can utilize pentoses such as L-arabinose and D-xylose. Transport of L-arabinose into the cell is performed by two inducible systems: low-affinity permease (Kₘ about 0.1 mM) encoded by araE and high-affinity (Kₘ 1 to 3 μM) system encoded by the araFG operon. The araF gene encodes a periplasmic binding protein (306 amino acids) with chemotactic receptor function, and the araG locus encodes an inner membrane protein. The sugar is metabolized by a set of enzymes encoded by the araBAD operon: an isomerase (encoded by araA gene), which reversibly converts the aldose to L-ribulose; a kinase (encoded by araB gene), which phosphorylates the ketose to L-ribulose 5-phosphate; and L-ribulose-5-phosphate-4-epimerase (encoded by araD gene), which catalyzes the formation of D-xylose-5-phosphate (Escherichia coli and Salmonella, Second Edition, Editor in Chief: F. C. Neidhardt, ASM Press, Washington D.C., 1996).

Most strains of E. coli grow on D-xylose, but a mutation is necessary for strain K-12 to grow on the compound. Utilization of this pentose is through an inducible and catabolite-repressible pathway involving transport across the cytoplasmic membrane by two inducible permeases (not active on D-ribose or D-arabinose), isomerization to D-xylulose, and ATP-dependent phosphorylation of the pentulose to yield D-xylulose 5-phosphate. The high-affinity (Kₘ 0.3 to 3 μM) transport system depends on a periplasmic binding protein (37,000 Da) and is probably driven by a high-energy compound. The low-affinity (Kₘ about 170 μM) system is energized by proton motive force. This D-xylose-proton-symport system is encoded by xylE gene. The main gene cluster specifying D-xylose utilization is xylAB(RT). The xylA gene encodes the isomerase (54,000 Da) and xylB gene encodes the kinase (52,000 Da). The operon contains two transcriptional start points, one of them is placed before xylB open reading frame. But it is not essential here. Since the low-affinity permease is specified by the unlinked xylE, the xylT locus probably codes for the high-affinity transport system and therefore should contain at least two genes (one for a periplasmic protein and one for an integral membrane protein) (Escherichia coli and Salmonella, Second Edition, Editor in Chief: F. C. Neidhardt, ASM Press, Washington D.C., 1996).

The introduction of above-mentioned E. coli genes coding for L-arabinose isomerase, L-ribulokinase, L-ribulose 5-phosphate 4-epimerase, xylose isomerase and xylulokinase in addition to transaldolase and transketolase allow a microbe, such as Zymomonas mobilis, to metabolize arabinose and xylose to ethanol (WO/9528476, WO98/50524). In contrast, Zymomonas genes encoding alcohol dehydrogenase (ADH) and pyruvate decarboxylase (PDH) are useful for ethanol production by Escherichia coli strains (Dien B. S. et al, Appl. Biochem. Biotechnol, 2000, 84-86:181-96; U.S. Pat. No. 5,000,000).

At present there is no report describing a process for producing L-amino acid by fermentation of a mixture of glucose and pentoses, such as arabinose and xylose.

### SUMMARY OF THE INVENTION

An object of the present invention is to provide a process for producing L-amino acids from a mixture of hexose sugar, such as glucose, and pentose sugars, such as xylose or arabinose, by culturing the L-amino acid-producing microorganism in a culture medium containing a mixture of sugars. A fermentation feedstock obtained from cellulosic biomass may be used as a carbon source for the culture medium. A microorganism capable of growth on the fermentation feedstock and efficient in production of L-amino acids may be used, using the fermentation feedstock consisting of xylose and arabinose along with glucose, as the carbon source.

It is an object of the present invention to provide a process for producing an L-amino acid, which comprises cultivating the L-amino acid-producing bacterium in a culture medium and collecting from the culture medium the L-amino acid, wherein the culture medium contains a mixture of glucose and pentose sugars.

It is a further object of the present invention to provide the process as described above, wherein the pentose sugars are arabinose and xylose.

It is a further object of the present invention to provide the process as described above, wherein the mixture of sugars is a feedstock mixture of sugars obtained from cellulosic biomass.

It is a further object of the present invention to provide the process as described above, wherein the L-amino acid-producing bacterium is the bacterium belonging to the genus Escherichia.

It is a further object of the present invention to provide the process as described above, wherein the L-amino acid-producing bacterium is modified to have an increased rate of pentose sugars utilization.

It is a further object of the present invention to provide the process as described above, wherein the L-amino acid to be produced is L-isoleucine.

It is a further object of the present invention to provide the process as described above, wherein the bacterium has enhanced expression of genes for isoleucine biosynthesis.

It is a further object of the present invention to provide the process as described above, wherein the L-amino acid to be produced is L-histidine.

It is a further object of the present invention to provide the process as described above, wherein the bacterium has enhanced expression of genes for histidine biosynthesis.

It is a further object of the present invention to provide the process as described above, wherein the L-amino acid to be produced is L-threonine.

It is a further object of the present invention to provide the process as described above, wherein the bacterium has enhanced expression of genes for L-threonine biosynthesis.

It is a still further object of the present invention to provide the process as described above, wherein the L-amino acid to be produced is L-tryptophan.

It is even a further object of the present invention to provide the process as described above, wherein the bacterium has enhanced expression of genes for L-tryptophan biosynthesis.

### DESCRIPTION OF THE PREFERRED EMBODIMENTS

The present inventors have found that known L-amino acid-producing strains could efficiently utilize pentose sugars along with glucose and produce L-amino acids in an amount comparable to the amount of L-amino acids produced by fermentation of glucose. Examples of L-amino acid-producing strains include a strain belonging to the genus Escherichia.

In other words, the present invention describes the use of recombinant strains of simple organisms for the production of L-amino acid from under-utilized sources of biomass, such as cellulose and hemicellulose, which represents a major portion of wood and inedible plant parts.

Thus the present invention has been completed.

The method for producing L-amino acids includes production of L-isoleucine by fermentation of a mixture of glucose and pentose sugars, such as arabinose and xylose. Also, the method for producing L-amino acids includes production of L-histidine by fermentation of a mixture of glucose and pentose sugars, such as arabinose and xylose. Also, the method for producing L-amino acids includes production of L-threonine by fermentation of a mixture of glucose and pentose sugars, such as arabinose and xylose. Also, the method for producing L-amino acid includes production L-tryptophan by fermentation of mixture of glucose and pentose sugars, such as arabinose and xylose. Such a mixture of glucose and pentose sugars used as a fermentation feedstock could be obtained from under-utilized sources of plant biomass.

The present invention will be explained in detail below.

In the present invention, “L-amino acid-producing bacterium” means a bacterium, having an ability to cause accumulation of L-amino acids in a medium when the bacterium of the present invention is cultured in the medium. The L-amino acid-producing ability may be imparted or enhanced by breeding. The term “L-amino acid-producing bacterium” used herein also means a bacterium, which is able to produce and cause accumulation of L-amino acids in a culture medium in an amount larger than a wild-type or parental strain, and preferably means that the microorganism is able to produce and cause accumulation in a medium of an amount not less than 0.5 g/L, more preferably not less than 1.0 g/L of a target L-amino acid. “L-amino acid” includes L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine and L-valine.

The term “a bacterium belonging to the genus Escherichia” means that the bacterium is classified as the genus Escherichia according to the classification known to a person skilled in the art of microbiology. Examples of microorganisms belonging to the genus Escherichia used in the present invention include Escherichia coli (E. coli).

Examples of L-amino acid-producing bacteria belonging to the genus Escherichia are described below.

L-isoleucine-producing bacteria

As bacteria belonging to the genus Escherichia having L-isoleucine-producing ability, E. coli strain AJ12919 (Japanese Patent Laid-open Publication No. 8-47397); E. coli strains VL 1892 and KX141 (VKPM B-4781) (U.S. Pat. No. 5,658,766); E. coli strains H-9146 (FERM BP-5055) and H-9156 (FERM BP-5056) (U.S. Pat. No. 5,695,972); E. coli strains H-8670 (FERM BP-4051) and H-8683 (FERM BP-4052) (U.S. Pat. No. 5,460,958); E. coli strain FERM BP-3757 (US5474918), and the like, are encompassed. The VKPM B-3996 strain in which the ilv operon is amplified (strain TDV5) is also a preferred L-isoleucine-producing bacterium (Hashiguchi K. et al, Biosci. Biotechnol. Biochem., 1999, 63(4), 672-9).

L-histidine-producing bacteria

As bacteria belonging to the genus Escherichia having L-histidine-producing ability, E. coli strain 24 (VKPM B-5945, RU2003677); E. coli strain 80 (VKPM B-7270, RU2119536); E. coli strains NRRL B-12116-B12121 (US4388405); E. coli strains H-9342 (FERM BP-6675) and H-9343 (FERM BP-6676) (US6344347); E. coli strain H-9341 (FERM BP-6674) (EP1085087); E. coli strain AI80/pFM201 (US6258554), and the like, are emcompassed.

L-threonine-producing bacteria

As bacteria belonging to the genus Escherichia having L-threonine-producing ability, E. coli strain TDH6/pVIC40 (VKPM B-3996) (U.S. Pat. No. 5,175,107, U.S. Pat. No. 5,705,371), E. coli strain NRRL-21593 (U.S. Pat. No. 5,939,307), E. coli strain FERM BP-3756 (U.S. Pat. No. 5,474,918), E. coli strains FERM BP-3519 and FERM BP-3520 (U.S. Pat. No. 5,376,538), E. coli strain MG442 (Gusyatiner et al., Genetika (in Russian), 14, 947-956 (1978)), E. coli strains VL643 and VL2055 (EP 1149911 A), and the like, are encompassed.

L-tryptophan-producing bacteria

As bacteria belonging to the genus Escherichia having L-tryptophan-producing ability, E. coli strains JP4735/pMU3028 (DSM10122) and JP6015/pMU91 (DSM10123) deficient in the tryptophanyl-tRNA synthetase coded by mutant trpS gene (U.S. Pat. No. 5,756,345); E. coli strain SV164 (pGH5) having serA allele freed from feedback inhibition by serine (U.S. Pat. No. 6,180,373); E. coli strains AGX17 (pGX44) (NRRL B-12263) and AGX6(pGX50)aroP (NRRL B-12264) deficient in the enzyme tryptophanase (U.S. Pat. No. 4,371,614); E. coli strain AGX17/pGX50,pACKG4-pps in which a phosphoenolpyruvate-producing ability is enhanced (WO97/08333, U.S. Pat. No. 6,319,696), and the like, are encompassed.

The above-mentioned L-amino acid-producing strains may be further modified for enhancement of pentose assimilation rate or for enhancement of L-amino acid biosynthetic ability by the wide scope of methods well-known for the person skilled in the art.

Pentose sugar utilization rate could be enhanced by amplification of pentose assimilation genes, such as araFG and araBAD genes for arabinose, and xylE and xylAB(RT) genes for xylose, or by mutations in glucose assimilation systems (PTS and non-PTS), such as ptsG mutations (Nichols N. N. et al, Appl. Microbiol. Biotechnol., 2001, July 56:1-2, 120-5).

Biosynthetic ability of the L-amino acid-producing bacterium may be further improved by enhancing expression of one or more genes which are involved in L-amino acid biosynthesis. Such genes include ilvGMEDA operon, which preferably comprises an ilvA gene encoding threonine deaminase substantially released from inhibition by L-isoleucine (U.S. Pat. No. 5,998,178), for L-isoleucine-producing bacteria. Also, such genes include a histidine operon, which preferably comprises hisG gene encoding ATP phosphoribosyl transferase for which feedback inhibition by L-histidine is desensitized (Russian patents 2003677 and 2119536) for L-histidine producing bacteria. Also, such genes include a threonine operon, which preferably comprises a gene encoding aspartate kinase—homoserine dehydrogenase for which feedback inhibition by L-threonine is desensitized (Japanese Patent Publication No. 1-29559), for L-threonine producing bacteria. And such genes include trpEDCBAoperon, which preferably comprises trpE gene encoding anthranilate synthase freed from feedback inhibition by L-tryptophan; serA gene freed from feedback inhibition by serine; pps gene supplying the common pathway of aromatic acids with phosphoenolpyruvate, for the L-tryptophan bacteriua. Also, the ability of a bacterium to produce L-tryptophan may be further improved by imparting the bacterium with a deficiency in enzymes utilizing L-tryptophan, which preferably comprises deficient tryptophanyl-tRNA synthetase coded by mutant trpS gene or deficient tryptophanase coded by mutant aroP gene.

The process of present invention includes a process for producing an L-amino acid, comprising the steps of cultivating the L-amino acid-producing bacterium in a culture medium, allowing production and accumulation of the L-amino acid in the culture medium, and collecting the L-amino acid from the culture medium, wherein the culture medium contains a mixture of glucose and pentose sugars. Also, the process of the present invention includes a process for producing L-isoleucine, comprising the steps of cultivating the L-isoleucine-producing bacterium in a culture medium, allowing production and accumulation of L-isoleucine in the culture medium, and collecting L-isoleucine from the culture medium, wherein the culture medium contains a mixture of glucose and pentose sugars. Also, the method of present invention includes a method for producing L-histidine, comprising the steps of cultivating the L-histidine-producing bacterium of the present invention in a culture medium, allowing production and accumulation of L-histidine in the culture medium, and collecting L-histidine from the culture medium, wherein the culture medium contains a mixture of glucose and pentose sugars. Also, the method of the present invention includes a method for producing L-threonine, comprising the steps of cultivating the L-threonine-producing bacterium of the present invention in a culture medium, allowing production and accumulation of L-threonine in the culture medium, and collecting L-threonine from the culture medium, wherein the culture medium contains a mixture of glucose and pentose sugars. Also, the method of the present invention includes a method for producing L-tryptophan, comprising the steps of cultivating the L-tryptophan-producing bacterium of the present invention in a culture medium, allowing production and accumulation of L-tryptophan in the culture medium, and collecting L-tryptophan from the culture medium, wherein the culture medium contains a mixture of glucose and pentose sugars.

A mixture of pentose sugars, such as xylose and arabinose, along with hexose sugar, such as glucose, can be obtained from under-utilized sources of biomass. Glucose, xylose, arabinose and other carbohydrates may be liberated from plant biomass by steam and/or concentrated acid hydrolysis, dilute acid hydrolysis, hydrolysis using enzymes, such as cellulase, or alkali treatment. When the substrate is cellulosic material, the cellulose may be hydrolyzed to sugars simultaneously or separately, and also fermented to L-amino acids. Since hemicellulose is generally easier to hydrolyze to sugars than cellulose, it is preferable to prehydrolyze the cellulosic material, separate the pentoses and then hydrolyze the cellulose by treatment with steam, acid, alkali, cellulases or combinations thereof to form glucose.

A mixture consisting of different ratios of glucose/xylose/arabinose is used in this study to approximate the composition of feedstock mixture of glucose and pentoses, which could potentially be derived from plant hydrolysates. Ratio of each pentose in the mixture varied from 12% to 50% of total carbohydrate content (see Example section).

In the present invention, cultivation, collection and purification of L-amino acids from the medium and the like may be performed in a manner similar to the conventional fermentation method wherein an amino acid is produced using a microorganism. A medium used for culture may be either a synthetic medium or a natural medium, so long as the medium includes a carbon source and a nitrogen source and minerals and, if necessary, appropriate amounts of nutrients which the microorganism requires for growth.

The carbon source may include various carbohydrates such as glucose, sucrose, arabinose, xylose and other pentose and hexose sugars, which the L-amino acid-producing bacterium could utilize as a carbon source. Glucose, xylose, arabinose and other carbohydrates may be a part of a feedstock mixture of sugars obtained from cellulosic biomass. In the present invenion, the ratio of glucose and pentose sugars is preferably 10:0.5-50, more preferably 10:1-25, most preferably 10:2-10. Pentose sugars suitable for fermentation by the present invention include, but are not limited to, xylose and arabinose. When xylose and arabinose are used as pentose sugars, the ratio of xylose and arabinose is preferably 1:0.5-10, more preferably 1:1-5, most preferably 1:2-3.

As the nitrogen source, various ammonium salts such as ammonia and ammonium sulfate, other nitrogen compounds such as amines, a natural nitrogen source such as peptone, soybean-hydrolysate and digested fermentative microorganism can be used. As minerals, potassium monophosphate, magnesium sulfate, sodium chloride, ferrous sulfate, manganese sulfate, calcium chloride, and the like can be used. Additional nutrients can be added to the medium, if necessary. For instance, if the microorganism requires L-threonine for growth (threonine auxotrophy), a sufficient amount of L-threonine can be added to the medium for cultivation.

The cultivation is performed preferably under aerobic conditions such as a shaking culture, and stirring culture with aeration, at a temperature of 20 to 40° C., preferably 30 to 38° C. The pH of the culture is usually between 5 and 9, preferably between 6.5 and 7.2. The pH of the culture can be adjusted with ammonia, calcium carbonate, various acids, various bases, and buffers. Usually, a 1 to 5-day cultivation leads to the accumulation of the target L-amino acid in the liquid medium.

After cultivation, solids such as cells can be removed from the liquid medium by centrifugation or membrane filtration, and then the target L-amino acid can be collected and purified by ion-exchange, concentration and crystallization methods.

### EXAMPLES

The present invention will be more concretely explained below with reference to the following non-limiting Examples.

### Example 1

Production of L-isoleucine by L-isoleucine producing bacterium in fermentation of mixture of glucose and pentoses.

The L-isoleucine-producing E. coli strain TDV5 was used as a strain for production of L-isoleucine by fermentation of a mixture of glucose and pentoses. Strain TDV5 is a derivative of E. coli strain TDH6/pVIC40 (VKPM B-3996), in which the ilv operon is additionally amplified (plasmid pMWD5) (Hashiguchi K. et al, Biosci. Biotechnol. Biochem., 1999, 63(4), 672-9).

To obtain seed culture, the strain was cultivated at 37° C. for 7 hours in LB broth and added to the fermentation medium in a ratio of 1/20 (v/v). 2 ml of seed culture were transferred into a 20×200 mm test tube with fermentation medium containing different sugars or mixtures thereof, and cultivated at 37° C. for 72 hours with a rotary shaker. After the cultivation, an amount of L-isoleucine which accumulated in the medium was determined by TLC. 10×15 cm TLC plates coated with 0.11 mm layers of Sorbfil silica gel without fluorescent indicator (Stock Company Sorbpolymer, Krasnodar, Russia) were used. Sorbfil plates were developed with a mobile phase: propan-2-ol:ethyl acetate: 25% aqueous ammonia: water=80:80:25:50 (v/v). Solution (2%) of ninhydrin in acetone was used as a visualizing reagent. The results (data of at least 3 independent experiments) are presented in Table 2.

The composition of the fermentation medium (g/l):

| | |
|---|---|
| Carbohydrate | 40.0 |
| (NH₄)₂SO₄ | 18.0 |
| K₂HPO₄ | 2.0 |
| MgSO₄ × 7H₂O | 1.0 |
| Thiamine HCl | 0.02 |
| CaCO₃ | 25.0 |

Glucose and magnesium sulfate are sterilized separately. CaCO₃ dry-heat are sterilized at 180° for 2 h. pH is adjusted to 7.0.

TABLE 2

| Carbohydrates | | | | |
|---|---|---|---|---|
| D-glucose | L-arabinose | D-xylose | OD₅₄₀ | L-isoleucine, g/l |
| 6% | — | — | 16.0 ± 2.3 | 15.0 ± 0.6 |
| — | 6% | — | 10.6 ± 1.3 | 5.2 ± 0.3 |
| — | — | 6% | 11.1 ± 0.4 | 8.4 ± 0.5 |
| 3% | 3% | — | 11.9 ± 2.0 | 9.5 ± 2.8 |
| 3% | — | 3% | 14.1 ± 0.3 | 12.8 ± 1.9 |
| 3% | 1.5% | 1.5% | 12.8 ± 1.5 | 11.8 ± 1.8 |
| 1.5% | 3% | 1.5% | 12.7 ± 1.2 | 10.4 ± 2.3 |
| 1.5% | 1.5% | 3% | 14.1 ± 1.1 | 11.8 ± 0.5 |

It can be seen from the Table 2, L-isoleucine producing E. coli strain TDV5 could efficiently utilize pentose sugars in the mixture with glucose and produce L-isoleucine in amount comparable to the amount of L-isoleucine produced by fermentation using glucose alone.

### Example 2

Production of L-histidine by L-histidine producing bacterium in fermentation of mixture of glucose and pentoses.

L-histidine-producing E. coli strain 80 was used as a strain for production of L-histidine by fermentation of a mixture of glucose and pentoses. E. coli strain 80 (VKPM B-7270) is described in detail in Russian patent RU2119536.

To obtain seed culture, the strain was grown on rotary shaker (250 rpm) at 27° C. for 6 hours in 40 ml test tubes (Ø 18 mm) containing 2 ml of L-broth with 3% glucose. Then the fermentation medium was inoculated with 2 ml (5%) of seed material. The fermentation was carried out on a rotary shaker (250 rpm) at 27° C. for 65 hours in 40 ml test tubes containing 2 ml of fermentation medium.

After the cultivation, the amount of accumulated L-histidine in the culture medium was determined by paper chromatography. Composition of the mobile phase is the following: butanol:acetate: water=4:1:1 (v/v). Solution (0.5%) of ninhydrin in acetone was used as a visualizing reagent. The results are presented in Table 3.

The composition of the fermentation medium (g/l):

| | |
|---|---|
| Carbohydrate | 100.0 |
| Mameno | 0.2 of TN |
| (soybean protein hydrolysate) | |
| L-threonine | 0.8 |
| (NH₄)₂SO₄ | 25.0 |
| K₂HPO₄ | 2.0 |
| MgSO₄ × 7H₂O | 1.0 |
| FeSO₄ × 7H₂O | 0.01 |
| MnSO₄ × 5H₂O | 0.01 |
| Thiamine HCl | 0.001 |
| Betaine | 2.0 |
| CaCO₃ | 6.0 |

Glucose, L-threonine and magnesium sulfate are sterilized separately. CaCO₃ dry-heat are sterilized at 110° C. for 30 min. pH is adjusted to 6.0 by KOH before sterilization.

TABLE 3

| Carbohydrates | | | | |
|---|---|---|---|---|
| D-glucose | L-arabinose | D-xylose | OD₄₅₀ | L-histidine, g/l |
| 10% | — | — | 33.8 | 13.0 |
| — | 10% | — | 30.5 | 14.2 |
| — | — | 10% | No growth | — |
| 5% | 5% | — | 29.0 | 13.6 |
| 5% | — | 5% | 32.6 | 7.8 |
| 5% | 2.5% | 2.5% | 28.9 | 10.2 |
| 5% | 1.25% | 3.75% | 28.0 | 6.6 |
| 5% | 3.75% | 1.25% | 29.0 | 13.9 |
| 2.5% | 3.75% | 3.75% | 25.7 | 9.0 |

It can be seen from the Table 3, L-histidine-producing E. coli strain 80 could efficiently utilize pentose sugars in the mixture with glucose and produce L-histidine in an amount comparable to the amount of L-histidine produced by fermentation using glucose alone.

### Example 3

Production of L-threonine by L-threonine producing bacterium in fermentation of mixture of glucose and pentoses.

L-threonine-producing E. coli strain TDH6/pVIC40 (VKPM B-3996) was used as a strain for production of L-threonine by fermentation of a mixture of glucose and pentoses. E. coli strain TDH6/pVIC40 is described in detail in U.S. Pat. No. 5,175,107.

To obtain a seed culture, the strain was grown on a rotary shaker (250 rpm) at 32° C. for 18 hours in 40 ml test tubes (Ø 18 mm) containing 2 ml of L-broth with 4% glucose. Then the fermentation medium was inoculated with 2 ml (5%) of seed material. The fermentation was carried out on a rotary shaker (250 rpm) at 32° C. for 24 hours in 40 ml test tubes containing 2 ml of fermentation medium.

After the cultivation, the amount of accumulated L-threonine in the medium was determined by TLC. Sorbfil plates (Stock Company Sorbpolymer, Krasnodar, Russia) were developed with a mobile phase: propan-2-ol:acetone: water: 25% aqueous ammonia=25:25:7:6 (v/v). A solution (2%) of ninhydrin in acetone was used as a visualizing reagent. The results (data of at least 3 independent experiments) are presented in Table 4.

The composition of the fermentation medium (g/l):

| | |
|---|---|
| Carbohydrates | 40.0 |
| (NH₄)₂SO₄ | 10.0 |
| K₂HPO₄ | 1.0 |
| MgSO₄ × 7H₂O | 0.4 |
| FeSO₄ × 7H₂O | 0.02 |
| MnSO₄ × 5H₂O | 0.02 |
| Thiamine HCl | 0.0002 |
| Yeast extract | 1.0 |
| CaCO₃ | 20.0 |

Glucose and magnesium sulfate are sterilized separately. CaCO₃ dry-heat are sterilized at 180° C. for 2 h. pH is adjusted to 7.0. Antibiotic is introduced into the medium after sterilization.

TABLE 4

| Carbohydrates | | | | |
|---|---|---|---|---|
| D-glucose | L-arabinose | D-xylose | OD₄₅₀ | L-threonine, g/l |
| 4% | — | — | 13.8 ± 0.5 | 14.1 ± 1.0 |
| — | 4% | — | 15.8 ± 0.2 | 14.9 ± 0.4 |
| — | — | 4% | 13.1 ± 0.1 | 16.6 ± 0.1 |
| 2% | 2% | — | 13.7 ± 0.2 | 15.9 ± 0.3 |
| 2% | — | 2% | 14.2 ± 0.5 | 14.5 ± 1.1 |
| 2% | 1% | 1% | 13.3 ± 0.4 | 15.8 ± 0.6 |
| 1% | 2% | 1% | 14.6 ± 0.3 | 16.6 ± 0.8 |
| 1% | 1% | 2% | 15.6 ± 0.7 | 12.4 ± 1.9 |

It can be seen from the Table 4, L-threonine-producing E. coli strain TDH6/pVIC40 could efficiently utilize pentose sugars in the mixture with glucose and produce L-threonine in amount comparable to the amount of L-threonine produced by fermentation using glucose alone.

### Example 4

Production of L-tryptophan by L-tryptophan-producing bacterium in fermentation of mixture of glucose and pentoses.

The tryptophan-producing E. coli strain SV164 (pGH5) was used as a strain for producing tryptophan by fermentation of a mixture of glucose and pentoses. The strain SV164 (pGH5) is described in detail in U.S. Pat. No. 6,180,373 or European patent 0662143.

To obtain seed culture, the strain was grown on a rotary shaker (250 rpm) at 37° C. for 18 hours in 40 ml test tubes (Ø 18 mm) containing 3 ml of L-broth with 4% glucose supplemented with 20 μg/ml of tetracycline (marker of pGH5 plasmid). Then 3 ml of fermentation medium containing tetracycline (20 μg/ml) in 20×200 mm test tubes was inoculated with 0.3 ml of the obtained cultures and cultivated at 37° C. for 48 hours with a rotary shaker at 250 rpm.

The composition of the fermentation medium is presented in Table 5.

TABLE 5

| Sections | Component | Final concentration, g/l |
|---|---|---|
| A | KH₂PO₄ | 1.5 |
| | NaCl | 0.5 |
| | (NH₄ )₂SO₄ | 1.5 |
| | L-Methionine | 0.05 |
| | L-Phenylalanine | 0.1 |
| | L-Tyrosine | 0.1 |
| | Mameno (total N) | 0.07 |
| B | Glucose | 40.0 |
| | MgSO₄ × 7H₂O | 0.3 |
| C | CaCl₂ | 0.011 |
| D | FeSO₄ × 7H₂O | 0.075 |
| | Sodium citrate | 1.0 |
| E | Na₂MoO₄ × 2H₂O | 0.00015 |
| | H₃BO₃ | 0.0025 |
| | CoCl₂ × 6H₂O | 0.00007 |
| | CuSO₄ × 5H₂O | 0.00025 |
| | MnCl₂ × 4H₂O | 0.0016 |
| | ZnSO₄ × 7H₂O | 0.0003 |
| F | Thiamine HCl | 0.005 |
| G | CaCO₃ | 30.0 |
| H | Pyridoxine | 0.03 |

Section A had pH 7.1 adjusted by NH₄OH. Each section was sterilized separately.

After the cultivation, the amount of tryptophan accumulated in the medium was determined by TLC. 10×15 cm TLC plates coated with 0.11 mm layers of Sorbfil silica gel without fluorescent indicator (Stock Company Sorbpolymer, Krasnodar, Russia) were used. Sorbfil plates were developed with a mobile phase: propan-2-ol:ethylacetate:25% aqueous ammonia: water=40:40:7:16 (v/v). A solution (2%) of ninhydrin in acetone was used as a visualizing reagent. Obtained data (data of at least 3 independent experiments) are presented in Table 6.

TABLE 6

| Carbohydrates | | | | |
|---|---|---|---|---|
| D-glucose | L-arabinose | D-xylose | OD₅₆₀ | L-tryptophan, g/l |
| 4% | — | — | 10.5 ± 0.1 | 4.7 ± 0.2 |
| — | 4% | — | 1.6 ± 0.1 | traces |
| — | — | 4% | 0.7 ± 0.1 | traces |
| 2% | 2% | — | 9.1 ± 1.0 | 5.2 ± 0.1 |
| 2% | — | 2% | 9.1 ± 0.2 | 3.8 ± 0.1 |
| — | 2% | 2% | 6.2 ± 1.1 | 0.9 ± 0.1 |
| 1.33% | 1.33% | 1.33% | 9.2 ± 0.3 | 4.4 ± 0.1 |
| 0.4% | 1.8% | 1.8% | 3.6 ± 0.2 | 3.8 ± 0.1 |

It can be seen from Table 6, L-tryptophan-producing E. coli strain SV 164 (pGH5) could efficiently utilize pentose sugars in the mixture with glucose and produce L-tryptophan in amount comparable to the amount of L-tryptophan produced by fermentation using glucose alone.

While the invention has been described with reference to preferred embodiments thereof, it will be apparent to one skilled in the art that various changes can be made, and equivalents employed, without departing from the scope of the invention. Each of the aforementioned documents, including the foreign priority document, RU 2003105269, is incorporated by reference herein in its entirety.

What is claimed is:

1). A process for producing an L-amino acid, which comprises cultivating the L-amino acid-producing bacterium in a culture medium resulting in production of the L-amino acid, and collecting the L-amino acid from the culture medium, wherein the culture medium contains a mixture of glucose and pentose sugars.

2). The process according to claim 1, wherein the pentose sugars are arabinose and xylose.

3). The process according to claim 2, wherein the mixture of sugars is a feedstock mixture of sugars obtained from cellulosic biomass.

4). The process according to claim 1, wherein the L-amino acid-producing bacterium is the bacterium belonging to the genus Escherichia.

5). The process according to claim 4, wherein the L-amino acid-producing bacterium is modified to have increased rate of pentose sugars utilization.

6). The process according to claim 1, wherein the L-amino acid is L-isoleucine.

7). The process according to claim 6, wherein the bacterium has enhanced expression of genes for L-isoleucine biosynthesis.

8). The process according to claim 1, wherein the L-amino acid is L-histidine.

9). The process according to claim 8, wherein the bacterium has enhanced expression of genes for L-histidine biosynthesis.

10). The process according to claim 1, wherein the L-amino acid is L-threonine.

11). The process according to claim 10, wherein the bacterium has enhanced expression of genes for L-threonine biosynthesis.

12). The process according to claim 1, wherein the L-amino acid is L-tryptophan.

13). The process according to claim 12, wherein the bacterium has enhanced expression of genes for L-tryptophan biosynthesis.
